(19) United States
(12) Patent Application Publication
(10) Pub. No.: US 2022/0079818 A1
(43) Pub. Date: Mar. 17, 2022

(54) Feminine Hygiene Kit for Sudden Menstrual Emergencies

(71) Applicant: Catrina Calvert, Miami, FL (US)

(72) Inventor: Catrina Calvert, Miami, FL (US)

(21) Appl. No.: 16/948,323

(22) Filed: Sep. 14, 2020

(51) Int. Cl. A61F 13/551; A61F 13/496; A61F 13/47; A61F 13/20; A61F 13/53

(52) U.S. Cl. CPC A61F 13/5514; A61F 13/496; A61F 13/47; A61F 2013/530364; A61F 13/55145; A61F 13/53; A61F 13/20

(57) ABSTRACT

The invention is a sanitary kit for feminine hygiene components to be used and stored in a pack for use during the sudden onset of menstruation. At least two different types of disposable absorbent articles for absorbing liquids, particularly body fluids such as menses, and a plurality of cleansing components for cleansing the body, clothing, or surrounding area after the discharge of menstrual fluids, are contained within the package and hygiene kit. These items could consist of menstruation briefs or panties, tampons or sanitary napkins, moistened sanitary towelettes or wiping objects, a compressed washcloth, and a cleansing liquid contained in a bottle. The packaging allows for easy storage, being in the form of a pack containing the items, with each unit or component sealed, packaged and identified. The preferred embodiment of the pack will take the form of a pouch with an opening and closure mechanism from which the items can be easily accessed or stored.

### BACKGROUND OF INVENTION

Many women experience the sudden, often unpredictable onset of their menstrual cycle while at work, when away from the home, or in a public space. During these times, it is crucial for women to be able to maintain their hygiene, privacy, and confidence quickly and easily. What would be required in these circumstances, is generally, the use of a sanitary pad or tampon, a change of underwear, and various other hygiene related items, such as a washcloth, moistened sanitary towelette and a cleaning agent such as hydrogen peroxide three percent to be used during emergencies and sudden menstruation.

These items are usually purchased separately and kept on hand in a woman's purse or handbag. In some cases, an adult woman enduring the onset of her menstrual cycle may also have to rush to various retail outlets in order to quickly purchase the required items needed to maintain her hygiene. This can be difficult and embarrassing, as leaving the office or a public space, to complete the sudden purchase of many hygiene products may put a woman's personal bodily experience on display for her colleagues and the general public surrounding her. Such an event can feel quite intrusive to the privacy of a woman enduring the sudden onset of menstruation. Therefore, a woman experiencing the sudden onset of menstruation is left in a conundrum, unless she has taken advanced steps to cater for this contingency. Such steps, which include purchasing and personally storing each required item, is time consuming and hard to maintain consistently on a month to month basis. There are also numerous threats to a woman's health and personal hygiene, if she is left unable to quickly access the right products, such as fresh underwear and sanitary pads, or tampons, as a result of discharges during menstruation.

There are currently no adequate hygiene products which combine all the required items necessary for use during menstruation, in one discreet, compact, simple, and easy to use packaging system. Such a system would cater to the complete needs of a woman during the sudden onset of menstruation, which are the maintenance of hygiene, treatment of discharges, fresh underwear, cleaning items, and packaging discreet enough to protect privacy, and effect easy storage or access.

### SUMMARY

Due to the kit-type-nature of the present system, the skilled individual studying this disclosure will recognize and necessarily note that each and every embodiment of the present invention need not match each object or aspect of the invention discussed below. Nor will each object or aspect of the invention be reflected in every claim. As a result, the features, systems, and assemblies noted and discussed in terms of their multiple aspects must be considered flexibly and not in a rigid lock-step analysis with any particular aspect or object.

The present invention comprises items necessary for the maintenance of feminine hygiene, in one compact, easy to store and easy to access container, with unique branded and packaging. The items stored in the package could be menstruation briefs or panties, tampons, sanitary napkins, a compressed washcloth, a feminine wipe, and a cleansing liquid for cleaning blood stains or other liquid stains. The invention caters to women who experience a sudden onset of menstruation, by allowing them to access all the products they need immediately, in a packaged form easily stored within a handbag or purse. The pack can also be stored discreetly in a desk drawer, closet, bathroom cabinet, or office cupboard and filing cabinet. The packaging is efficiently small enough to allow for convenient storage, and the packaging design is discreet enough to prevent public awareness that a woman is experiencing menstruation. The packs can also be left in regularly accessed public spaces, such as an office desk drawer, or a locker. This allows a woman to access all the products she needs during menstruation quickly, easily, and without encountering any invasion of privacy.
(Drawings included on the following page)

### BRIEF DESCRIPTION OF THE DRAWINGS

FIG. 1 shows a sectional view of one embodiment of the closed pack;

FIG. 2A shows a sectional view of another embodiment of the pack with its flap opened and inner contents;

FIG. 2B shows a sectional view of still another embodiment of the open pack with its contents and the storage of sanitary napkins instead of tampons;

FIG. 2C shows a sectional view of still another embodiment of the open pack with its contents, and both sanitary napkins and tampons displayed within it.

FIG. 3A shows a sectional view of still another embodiment of the open pack, with its contents displayed as stored inside;

FIG. 3B shows a sectional view of another embodiment of the open pack, without any contents, and two flaps open;

FIG. 3C shows a sectional view of another embodiment of the open pack, without any contents, and one flap open.

FIG. 3D shows a sectional view of another embodiment of the open pack, being buttoned down and closed.

FIG. 4 shows a sectional view of all the contents stored in the pack, both compressed and expanded, as they would appear outside the pack, in addition to a sectional view of the open pack without any contents inside.

FIG. 5 shows an aerial view of the pack without any contents.

FIG. 6 shows a simple embodiment of the button to which the flaps of the pack may be fastened.

### DETAILED DESCRIPTION OF DRAWINGS

The terminology used herein is for the purpose of describing particular embodiments only and is not intended to be limiting. As used in the specification and the appended claims, the singular forms “a,” “and” and “the” include plural references unless the context clearly dictates otherwise.

Any ranges given either in absolute terms or in approximate terms are intended to encompass both, and any definitions used herein are intended to be clarifying and not limiting. Notwithstanding the approximations provided, the numerical ranges and parameters setting forth the broad scope of the invention are approximations. This Specification contemplates various hygiene items, compressed into a small pack (100) made from either leather, plastic or other fabrics (such as cotton or wool) or any other suitable material. The dimensions of the pack (100) may vary, however one embodiment will have measurements of approximately 14 cm by 14 cm (length by height), and with a width of 5 cm, though this may vary with manufacturing processes. To be clear, the invention claimed is the distribution of various feminine hygiene items “in a generic form”, packaged in a single packet and distributed all together under one form of discreet packaging. The pack (100) could contain important hygiene items such as but not limited to menstruation briefs (101), tampons (102), cleansing liquid bottle (103), moistened sanitary wiping objects (104), a washcloth (105) and sanitary napkins (106). The packaging will also be in the form of a bag or pouch and an opening in which the items stored within can be accessed from.

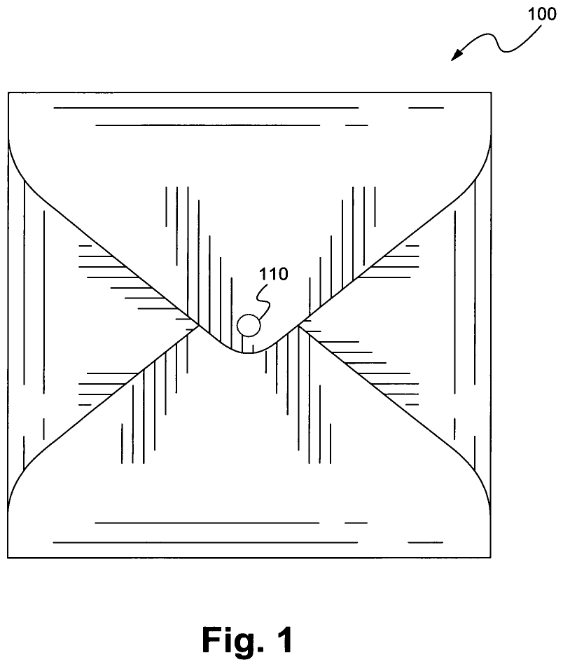

FIG. 1 shows an embodiment of the closed pack (100) of a rectangular shape, with a flap/opening along the top with a hole (109), which is buttoned down and closed around the center of the pack (100), through the use of a small opening which is fastened to a button (110) located at the center of the pack (100). The items are stored inside the pack (100), and the pack (100) has sufficient volume, width and length to accommodate all items within it.

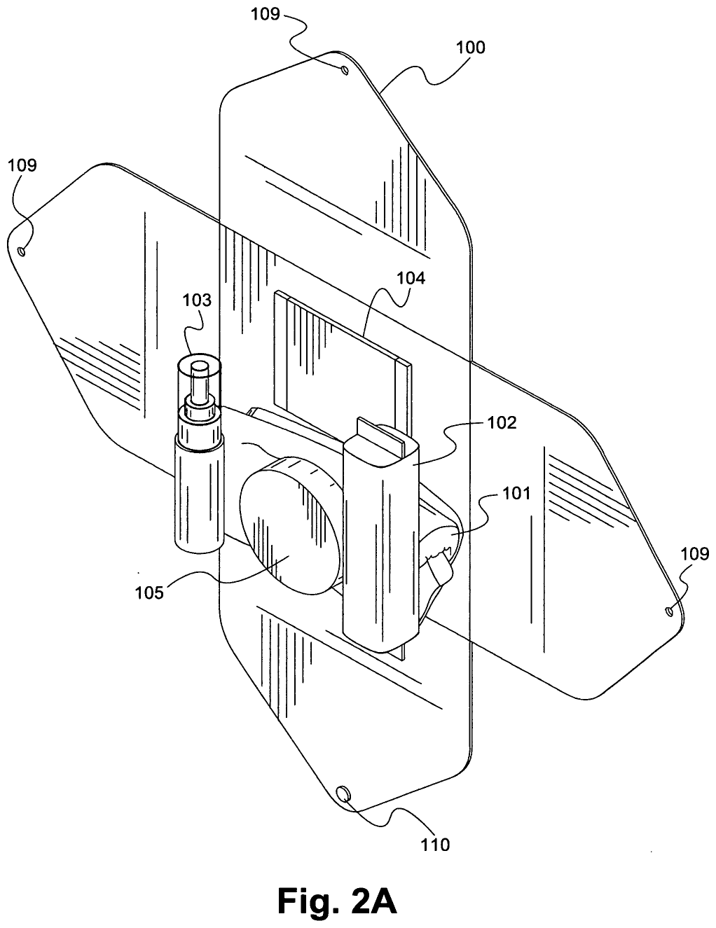

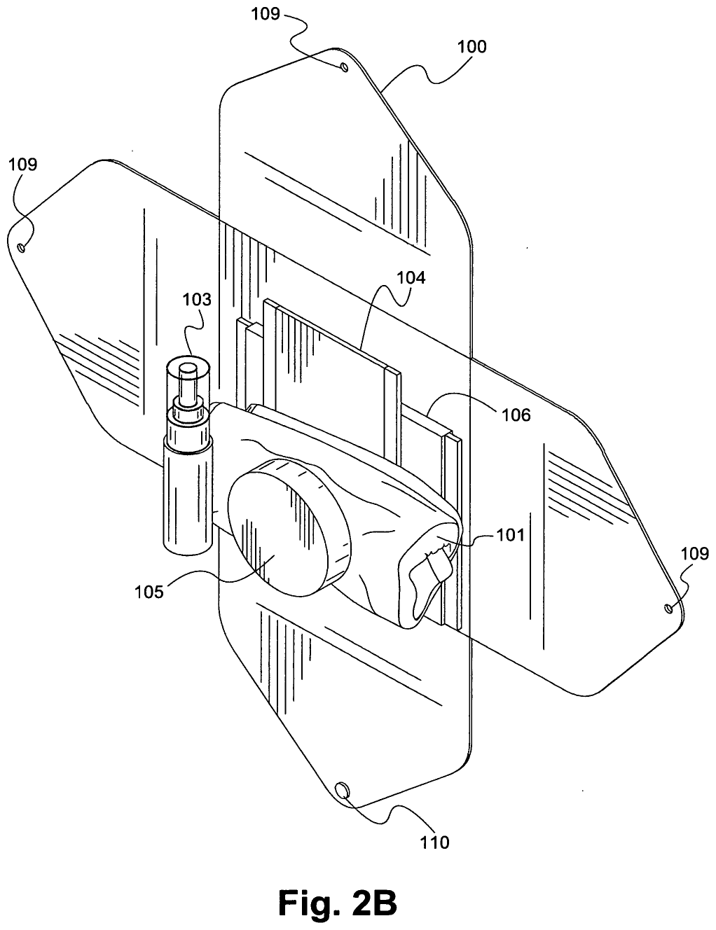

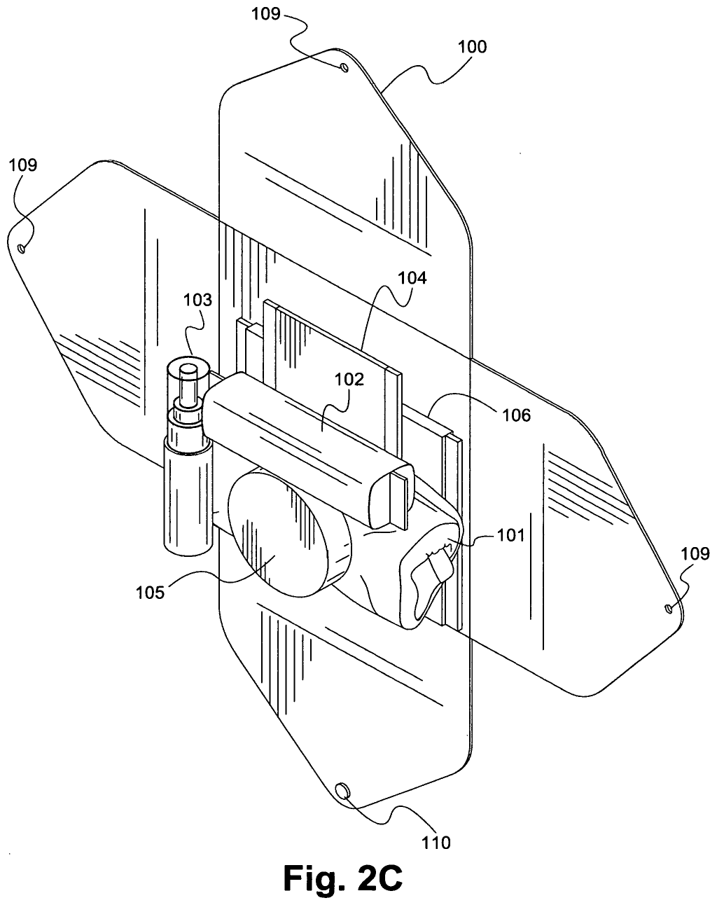

FIG. 2A shows an embodiment of the pack (100) opened with an angled vantage point from above the pack (100) and its opening, and the contents of the pack (100) are displayed as they could potentially be stored and compressed within the pack (100), with the menstruation briefs (101), the tampons (102), the cleansing liquid bottle (103), the moistened sanitary wiping objects (104), and the washcloth (105) stored within the pack (100) as shown in FIG. 2A. One embodiment of the menstruation briefs (101), if included, may come in various sizes as is suitable. The menstruation briefs (101) can be created from any suitable materials, including bamboo fiber, spandex, cotton or other cloth materials, and will be water/moisture resistant to reinforce the function of the tampon (102) or sanitary napkin (106), and prevent leakage or stains. The tampon (102) may be made from cotton or other suitable materials, and its dimensions will vary depending on suitability. FIG. 2B shows the same pack (100) from the same vantage point as in FIG. 2A, with the menstruation briefs (101), the Sanitary Napkins (106) instead of the tampons (102), the cleansing liquid bottle (103), the moistened sanitary wiping objects (104), and the washcloth (105) stored within the pack (100) as shown in FIG. 2A. The sanitary napkin (106) may be made from cotton or other suitable materials as well. The sanitary napkin may have various measurements and dimensions depending on suitability. FIG. 2C shows the pack (100) from the same vantage point as in FIGS. 2A and 2B, but with all contents, including both sanitary napkins and tampons, stored within the pack (100)-(106).

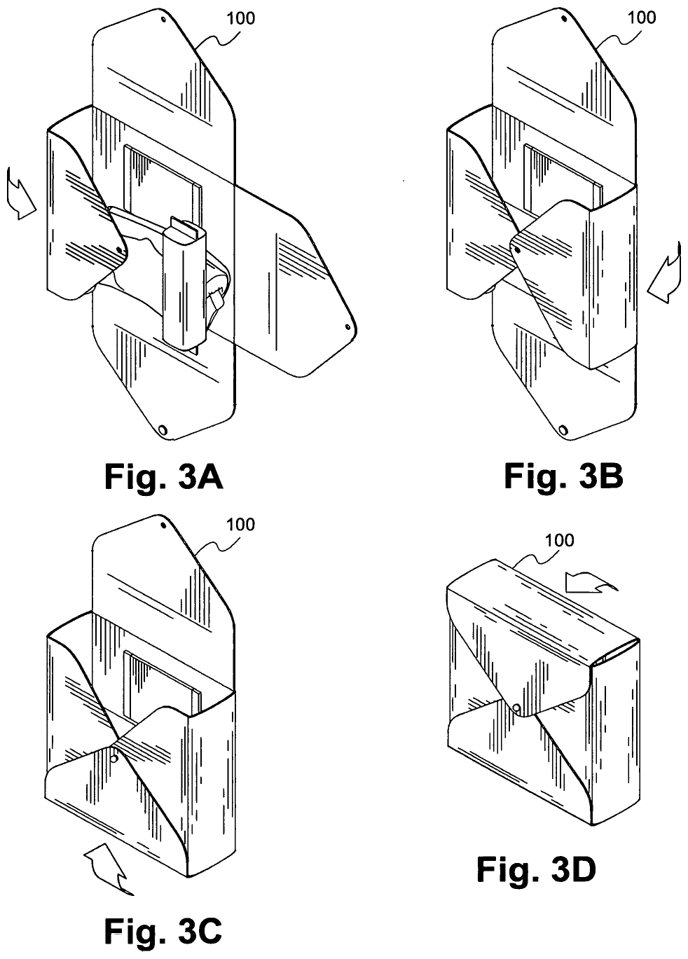

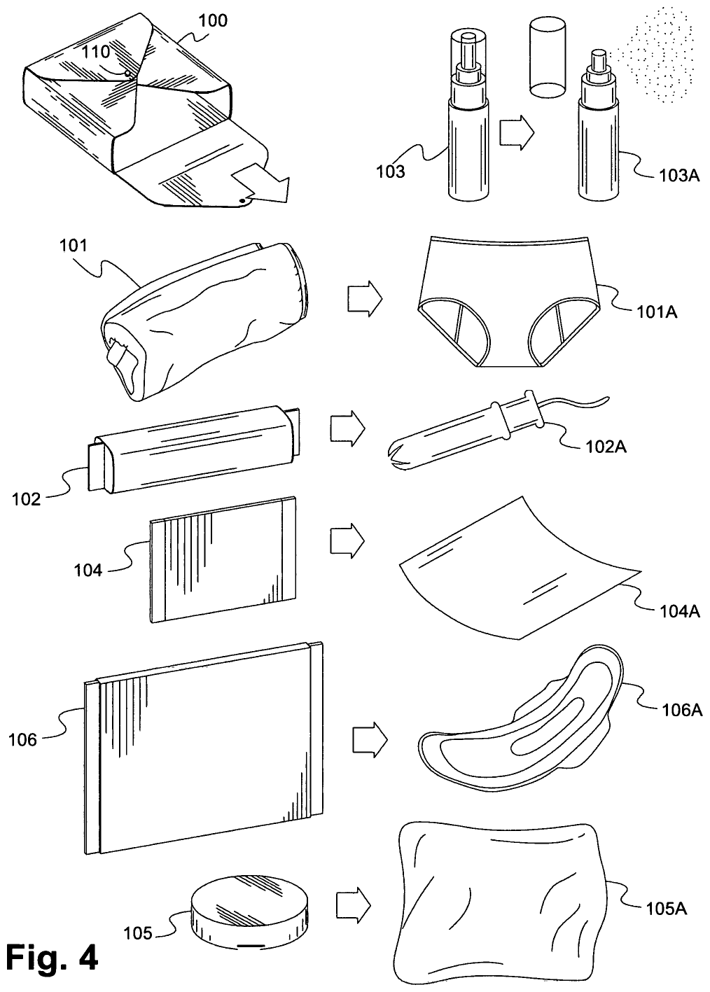

FIG. 3A-3D show an embodiment of the open pack (with the flap open) (100) from an angled vantage point above the pack (100), with and without any contents (101)-(106) inside.

FIG. 4. shows an embodiment of the opened and emptied pack (100) alongside a drawing of all the contents described and shown in previous drawings in both a compressed (101)-(106) and expanded form (101A)-(106A).

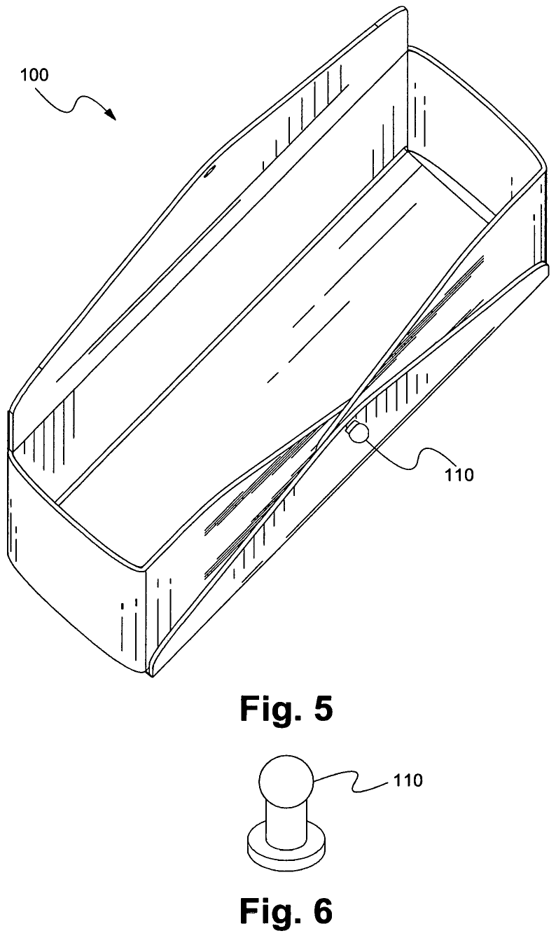

FIG. 5 shows an embodiment of the open pack (100) (with the flap open) (100), from an angled vantage point directly above the pack (100), without any of its contents displayed, to provide a view of the empty pack (100) by itself.

FIG. 6 shows a simple embodiment of the button (110) to which the flaps of the pack (100) may be fastened through their button holes (109). The button may come in various suitable forms, and velcro may also be used to help fasten the pack (100) more appropriately.

The invention is claimed as:

1. An emergency hygiene package for the storage and packaging of items to be used by women during menstruation, in the form of a bag or pouch with an opening in which the items stored within can be accessed from, and the packaging material may be made from any suitable material, such as leather, plastic or cloth.

2. The hygiene kit in claim 1, will contain various items used for menstrual hygiene which can include but are not limited to, menstruation briefs or panties, tampons, sanitary napkins, a cleansing liquid, moistened sanitary wiping objects and a washcloth, and this may also include a further array of personal hygiene products, associated with the overall function and purpose of the invention.

3. The contents of the items described in claim 2 may consist of any suitable materials, and for the purpose of providing examples and embodiments of their form, they are described with general specifications in claim 4 onwards.

4. The menstruation briefs or panties in claim 2 may come in various sizes, and the briefs can be created from any suitable materials, including bamboo fiber, spandex, or cotton or other cloth materials, and they will be water/moisture resistant to reinforce the function of the tampon, and prevent leakage or stains.

5. The tampons in claim 2 may be made from cotton or other suitable materials, and their dimensions will vary depending on suitability.

6. The sanitary napkins described in claim 2 may be made from cotton or other suitable materials as well, and the sanitary napkin may have various measurements and dimensions depending on suitability.

7. The cleansing liquid described in claim 2, may consist of hydrogen peroxide, or other suitable liquids, and as an example, this may come in a packaged bottle of 15 ml Hydrogen peroxide, or in other suitable quantities or forms.

8. The moistened sanitary wiping objects described in claim 2 may come in various forms as is suitable, and they may be composed of ingredients such as polyhexamethylene biguanide aloe glycerin, alcohol, or any other suitable material for such disinfecting wipes, and they may be made from any suitable materials such as non-woven fabrics, viscose or other cloth materials.

9. The compressed washcloth can be composed of any suitable material with drying effect, such as cotton or other suitable materials.

10. The pack may further comprise additional elements, for example, a pack may also include instructions for use of the items stored within it which would be affixed to packaging material or can be included as a package insert.
